(19) United States
(12) Patent Application Publication
(10) Pub. No.: US 2013/0164847 A1
(43) Pub. Date: Jun. 27, 2013

(54) USE OF A PROTEIN TYROSINE KINASE INHIBITOR FOR INDUCING THE DIFFERENTIATION OF MESENCHYMAL STEM CELLS INTO CARDIOGENIC CELLS

(71) Applicants: Ki-Chul Hwang, Seoul (KR); Yang-Soo Jang, Seoul (KR)

(72) Inventors: Ki-Chul Hwang, Seoul (KR); Yang-Soo Jang, Seoul (KR)

(73) Assignee: PHARMICELL. CO., LTD., Seoul (KR)

(21) Appl. No.: 13/822,758

(22) PCT Filed: Aug. 17, 2011
(86) PCT No.: PCT/KR11/06020

§ 371 Date: Mar. 13, 2013

(30) Foreign Application Priority Data
Sep. 16, 2010 (KR) 10-2010-0091189

(51) Int. Cl. C07D 239/94

(52) U.S. Cl. CPC C07D 239/94
USPC 435/377; 544/293

(57) ABSTRACT

The present invention pertains to methods of using protein tyrosine kinase (PTK) inhibitors for inducing cell differentiation of mesenchymal stem cells into cardiac cells, and a pharmaceutical composition comprising the differentiated cardiac cells thereof to treat heart diseases. The transplantation of mesenchymal stem cells to the heart provides immunological and functional improvements, but does not provide electrical stability. However, the mesenchymal stem cells treated with PTK inhibitors are induced to be differentiated into cardiogenic cells to provide electrical stability as the electromechanical integration with host heart tissue is improved, and it is thus possible to effectively treat cardiac diseases such as cardiac infarction, cardiac insufficiency, arrhythmia and the like.

### TECHNICAL FIELD

The present invention pertains to a method of using protein tyrosine kinase (PTK) inhibitors for inducing differentiation of mesenchymal stem cells into cardiogenic cells, and a pharmaceutical composition comprising induced differentiation of mesenchymal stem cells into cardiogenic cells for treatment of heart diseases.

### BACKGROUND ART

Although a numerous variety of cell types have been considered as a therapeutic candidate for stem cell treatment of ischemic heart, there has been no success in finding an ideal cell type in terms of electromechanical integration (Segers, V. F. & Lee, R. T. Nature 451, 937-942 (2008)). In reality, not only is differentiation of intact mesenchymal stem cells (MSCs) into cardiogenic cells rarely observed, but also the ability of electromechanical integration between transplanted stem cells and cardiogenic cells is uncertain (Chang, M. G., et al. Circulation 113, 1832-1841 (2006)).

In fact, the present inventors previously performed many experiments (Song, S. W., et al. Stem Cells 27, 1358-1365 (2009); Chang, W., et al. Stem Cells 27, 2283-2292 (2009); Song, H., et al. Stem Cells 25, 1431-1438 (2009)) to increase the merits of mesenchymal stem cells and tried to induce improvements of survival rates of transplanted cells or of the functions of infracted heart, yet have realized that transplantation of mesenchymal stem cells cannot reduce the rate of incidences of sudden death after the infarction. The inability to increase survival rates despite the improved functions is because there exists a lack of electromechanical integration between the transplanted stem cells and myocardium due to heterology between them.

### DISCLOSURE

### Technical Problem

The present invention intends to provide a method for inducing differentiation of mesenchymal stem cells into cardiogenic cells which can establish electromechanical integration with myocardium after transplantation of the mesenchymal stem cells as a cell therapy product that can be utilized for cardiac infarction, cardiac insufficiency, arrhythmia and the like.

### Technical Solution

In order to avoid heterology between transplanted cells and myocardium, it may be necessary to adjust mesenchymal stem cells to exhibit cardiogenic properties before the transplantation of the mesenchymal stem cells to an infarcted heart, and it is expected to bring enhancement of contractile function as well as electrical stability.

Based on this observation, the present inventors continued to investigate ways to induce mesenchymal stem cells into cardiogenic cells. As a result, the inventors found out that the protein tyrosine kinase (PTK) inhibitor has the ability to induce mesenchymal stem cells to differentiate into cardogenic cells. It is demonstrated in the following examples that the modified mesenchymal stem cells of rats by the PTK inhibitor in ex vivo differentiated into cardiogenic cells comparing to non-modified, intact mesenchymal stem cells.

Thus, the present invention provides a method of using PTK inhibitors to induce differentiation of mesenchymal stem cells into cardiogenic cells, a method of inducing differentiation of mesenchymal stem cells into cardiogenic cells wherein the mesenchymal stem cells are treated with PTK inhibitors, and a composition for inducing differentiation of PTK inhibitor treated mesenchymal stem cells into cardiogenic cells

In the present invention, the type of PTK inhibitors used for inducing differentiation of mesenchymal stem cells into cardiogenic cells is not particularly limited.

In a specific example, PTK inhibitors may be a compound of Formula I.

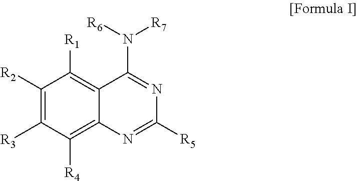

In the above formula, R₁, R₂, R₃, R₄, R₅ and R₆ are each independently H, C₁₋₁₂ alkyl, C₁₋₁₂ alkoxy, hydroxyl, carboxyl or halogen atom and R₇ is C₆₋₁₂ aryl, either unsubstituted or substituted with more than one substituents selected from the group consisting of C₁₋₁₂ alkyl, C₁₋₁₂ alkoxy, hydroxyl, carboxyl and halogen atom.

Here described “substitution” refers to more than one hydrogen atoms being replaced with more than one non-hydrogen atoms, provided that it satisfies valence requirements and must produce a chemically stable compound by the substitution. In the present application, unless explicitly stated as “unsubstituted”, it should be interpreted that all substituents are able to be substituted or unsubstituted. For example, R₁ to R₆ can be substituted again by more than one substituents defined above.

“Alkyls” generally represent straight or branched chains of saturated hydrocarbons with specified number of carbon atoms (for example, 1 to 12 carbon atoms). Examples of alkyls include but are not limited to methyl, ethyl, n-propyl, i-propyl, n-butyl, s-butyl, i-butyl, t-butyl, pent-1-yl, pent-2-yl, pent-3-yl, 3-methylbut-1-yl, 3-methylbut-2-yl, 2-methylbut-2-yl, 2,2,2,-trimethyleth-1-yl, n-hexyl, n-heptyl and n-oxytyl as such.

“Alkoxy” refers to alkyl-O— and the alkyl is defined above. Examples of alkoxy include but are not limited to methoxy, ethoxy, n-propoxy, n-butoxy, s-butoxy, t-butoxy, n-pentoxy, and s-pentoxy as such. The alkoxy may be attached to a parent group or to a substrate at any ring atom, provided that the attachment does not violate valence requirements Likewise, the alkoxy groups may include more than one non-hydrogen substituents, provided that the substitution does not violate valence requirements.

“Carboxyl” refers to —C(O)OH divalent radical. In the present application, (O) represents oxygen being double bonded to carbon or sulfur atoms.

“Aryl” refers to uni- and divalent aromatic base including 5-membered and 6-membered mono cyclic aromatic base respectively which they comprise 0 to 4 heteroatoms independently selected from nitrogen, oxygen and sulfur. When the aryl includes more than one heteroatom, it is also referred to as “heteroaryl.” Examples of monocyclic aryl include but are not limited to phenyl, pyrroline, furanyl, thiophenyl, thiazolyl, isothiazolyl, imidazolyl, triazolyl, tetrazolyl, pyrazolyl, oxazolyl, isoxazolyl, pyridinyl, pyrazinyl, pyridazinyl, and pyrimidinyl as such. Additionally, aryls also include bicylclic and tricyclic bases as such, including the above defined 5-membered and 6-membered fused rings. Examples of polycyclic aryls include, but are not limited to naphtyl, biphenyl, anthracenyl, pyrenyl, carbazolyl, benzoxazolyl, benzodioxazolyl, benzothiazolyl, benzoimidazolyl, benzothiopeneyl, quinolinyl, isoquinolinyl, indolyl, benzofuranyl, purinyl, and indolizinyl as such. The above aryls may be attached to a parent group or to a substrate at any ring atom, provided that the attachment does not violate valence requirements. Likewise, the aryl groups may include more than one non-hydrogen substituents, provided that the substitution does not violate valence requirements.

In a specific example, R₁, R₂, R₃, R₄, R₅ and R₆ are each independently H, C₁₋₄ alkyl or C₁₋₄ alkoxy and,

R₇ may be phenyl, either unsubstituted or substituted with more than one substituents selected from C₁₋₄ alkyl, C₁₋₄ alkoxy, hydroxyl, carboxyl or halogen atom.

In another specific example, R₁, R₂, R₃, R₄, R₅ and R₆ are each independently H, or C₁₋₄ alkoxy and,

R₇ may be phenyl, either unsubstituted or substituted with C₁₋₄ alkyl, C₁₋₄ alkoxy, or halogen atom.

In another specific example, PTK inhibitors may be N-(3-bromophenyl)-6,7-diethoxyquinazoline-4-amine.

Furthermore, in the present invention, the type of mesenchymal stem cells used for being induced to differentiate into cardiogenic cells is not particularly limited. In the present invention, the mesenchymal stem cells are used regardless of where they are derived from. Mesenchymal stem cells can be obtained from the known sources of mesenchymal stem cells such as bone marrow, tissues, embryo, cord blood, blood or body fluid. Animal subjects from which bone marrow and tissues as such are collected may be mammals. When the said animal is humans, bone marrow and tissues as such may be derived from a patient himself to whom the mesenchymal stem cells which are induced to differentiate into cardiogenic cells by being treated with the composition of the present invention would be administered as a cell therapy product, or it may be derived from other persons. The method of obtaining mesenchymal stem cells from the known sources of mesenchymal stem cells is well known to a person skilled in the art.

Meanwhile, a method of treating mesenchymal stem cells with PTK inhibitors is not particularly limited. For a fixed time, mesenchymal stem cells are brought into contact with PTK inhibitors so that PTK within the mesenchymal stem cells is inhibited. In a specific example, mesenchymal stem cells may be cultured in a medium which contains PTK inhibitors as a way to treat mesenchymal stem cells with PTK inhibitors.

The concentration of PTK inhibitors for treating mesenchymal stem cells may vary depending on the specific type of PTK inhibitors, the time period it takes to treat mesenchymal stem cells, and the extent of the differentiation requirement into cardiogenic cells and the like. In a specific example, the concentration of PTK inhibitors can be the range of 0.01 to 100 μm.

Considering the time generally required for inducing differentiation of mesenchymal stem cells, culturing in a medium containing PTK inhibitors can be performed for 5 to 15 days but it is not limited. The time period for treating mesenchymal stem cells with PTK inhibitors may change depending on the type and the concentration of inhibitors used.

In the present invention, “cardiogenic cells” include myocardial cells as well as cells in the process of becoming myocardial cells from the differentiation-induced mesenchymal stem cells. The present specification uses “cardiogenic cells” and “myocardial cells” interchangeably. In the present invention, cardiogenic cells differentiated by differentiation-induced, PTK inhibitor treated mesenchymal cells express cardiac specific markers. The cardiogenic cells yielded by the method according to the present invention may show an increased expression of cardiac specific markers compared to that of the mesenchymal stem cells. Cardiac specific markers may be selected from the group consisting of, but not limited to, cTnT (cardiac troponin T), MLC (myosin light chain) and MHC (myosin heavy chain). Furthermore, the expression of Cx43 (connexin 43) by the above cardiogenic cells may have increased compared to that of the mesenchymal stem cells. Additionally, the above cardiogenic cells may have increased expression of Ca²⁺ homeostasis-related proteins compared to that of the mesenchymal stem cells. The Ca²⁺ homeostasis-related proteins may be, but are not limited to, SERCA 2a or LTCC.

The present invention also provides a composition for inducing differentiation of mesenchymal stem cells into cardiogenic cells, wherein the compositions includes PTK inhibitors. As explained earlier, the type of PTK inhibitors used in the present invention is not particularly limited. In a specific example, the PTK inhibitors may be a compound of Formula I. Specific examples of the compound of Formula I are the same as explained above. The said composition may include a medium that is generally used for culturing mesenchymal stem cells. Examples of the medium include, but are not limited to, MEM-alpha (Minimum Essential Medium alpha), MSCGM (Mesenchymal Stem Cell Growth Medium), and DMEM (Dulbecco's Modified Eagle's Medium) as such.

Alternatively, the composition for inducing differentiation of mesenchymal stem cells into cardiogenic cells, wherein the composition includes PTK inhibitors can be introduced into the body, separately from mesenchymal stem cells. In other words, either before or after administering the mesenchymal stem cells, or simultaneously, the composition that includes the PTK inhibitors may be administered. In this case, the above composition may include a known pharmaceutically acceptable carrier for administering the PTK inhibitors.

Furthermore, the present invention provides a pharmaceutical composition for treating heart diseases, wherein the composition includes cardiogenic cells from induced differentiation of mesenchymal stem cells. Such pharmaceutical compositions for treating heart diseases can be effectively used for, but not limited to, heart diseases such as cardiac infarction, cardiac insufficiency, arrhythmia and the like. The above pharmaceutical composition may additionally include a pharmaceutically known carrier used for transplantation of stem cells. Additionally, the effective does of the above cardiogenic cells may be from 1×10⁴ and 1×10⁸ cells/kg. However, the capacity of the cells can be appropriately increased depending on a patient's weight, age, sex as well as the extent of lesions. The formulation according to the present invention can be applied to the body either parenterally or through topical administration. For these purposes, the active ingredients are suspended or dissolved in a pharmaceutically acceptable carrier in the usual manner, wherein utilizing aqueous carrier is preferred.

### Advantageous Effects

The transplantation of mesenchymal stem cells to the heart provides immunological and functional improvements, but does not provide electrical stability. However, the mesenchymal stem cells treated with PTK inhibitors are induced to be differentiated into cardiogenic cells to provide electrical stability as the electromechanical integration with host heart tissue is improved, and it is thus possible to effectively treat cardiac diseases such as cardiac infarction, cardiac insufficiency, arrhythmia and the like.

### DESCRIPTION OF DRAWINGS

FIG. 1 is a graph showing the incidences of sudden deaths of control (n=12), sham-administered rats (n=27) and MSCs-transplanted rats (n=19) for 11 days after damage and treatment.

FIG. 2 demonstrates the result of TTC stain for evaluating viability of myocardial tissue and for measuring the size of the myocardial infarction.

FIG. 3 demonstrates the result of Masson's trichrome stain for investigating interstitial fibrosis within transplanted cells.

FIG. 4 demonstrates the result of TUNEL assay for counting the numbers of cell death caused by apoptosis.

FIG. 5 demonstrates the result of H& E stain for measuring inflammatory cell infiltration.

FIG. 6 shows activity map and real images of infarcted heart.

FIG. 7 demonstrates action potential of control, sham-administered, MSCs- and CPMs-transplanted hearts.

FIG. 8 is a graph showing the recorded action potential durations (APDs) of the part where mesenchymal stem cells are transplanted.

FIG. 9 shows ectopic beat within the border zone around sham-administered myocardium.

FIG. 10 is a graph showing a localized conduction velocity (CV) of sham-administered heart and MSCs-transplanted heart.

FIG. 11 shows the result of electrical vulnerability test using the burst pacing protocol.

FIG. 12 shows sequential voltage map of ventricular tachycardia (VT) of MSC-transplanted area.

FIG. 13 shows the result of sandwich ELISA where differentiation into myocardial cells increases depending on the concentration of compound #23 treated.

### BEST MODE

The advantages and characteristics of the present invention, as well as the mode of achieving them, will be clarified by the detailed descriptions in the following embodiments. However, the present invention is not limited by the embodiments disclosed below, but is manifested in various forms. The present examples are provided only for the purpose of completing the disclosure of the present invention, and to teach the scope of the present invention completely to the person of ordinary skill in the art to which the present invention pertains. The present invention is defined solely by the scope of its claims.

### EXAMPLES

### Separating and Culturing rat MSCs

MSCs, obtained from (approximately 100 g of) the femoral and tibial bone marrow aspirate of a 4-week-old male Sprague-Dawley laboratory rat [/plural correct?], were cultured in 10 ml of DMEM-low glucose medium supplemented with 10% FBS and 1% antibiotic-penicillin/streptomycin solution (Invitrogen). The collected medium was centrifuged at 1,600 rpm for 5 minutes and resuspended in MSC medium, followed by 30 minutes of Percoll density gradient centrifugation at 1,600 rpm in Ficoll-Paque™ PLUS (GE Healthcare Life Sciences). After centrifugation, mononuclear myelocytes were collected from the middle interface and washed twice with PBS, then resuspended in 10% FBS-DMEM, and plated on a 100 cm² flask. The condition for the culture was maintained at 37° C. in moist air comprising 5% CO₂. After 72 hours, non-adherent cells were dropped from the flask and adherent cells were thoroughly washed twice with PBS. The product was treated with fresh MSC medium, which was replaced every three days, to generate the MSCs. The characteristics of MSCs were verified via immunophenotyping. The cells were labeled with respect to the various markers conjugated with fluorescent antibodies (CD14, CD34, CD71, CD90, CD105 and ICAM-1; Santacruz Biotechnology) and were analyzed via flow cytometry and immunofluorescence.

### Ex Vivo Differentiation of rat MSCs by the Treatment of PTK Inhibitor

For the second subculture, MSCs were dispensed on a 60 mm plate at 2×10⁵ cells/ml using the identical medium as above, treated with N-(3-bromophenyl)-6,7-diethoxyquinazoline-4-amine (Sigma), a protein tyrosine kinase (PTK) inhibitor, at a final concentration of 1 μM or 10 μM. The cells were cultured for 9 days, wherein the medium was replaced once every three days with a fresh medium containing the aforementioned compound.

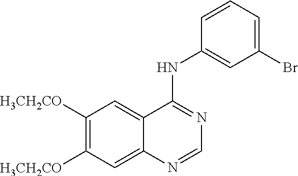

### Separating and Culturing Ventricular Myocardial Cells of Newborn Rats

Myocardial cells were obtained from the heart of a newborn Sprague-Dawley laboratory rat. In order to reduce erythrocytes, the separated heart tissues were washed with Dulbecco's phosphate-buffered saline solution (pH 7.4 Gibco BRL, New York). The heart was incised with micro-incision scissors to yield pieces of approximately 0.5 mm³ in size, which were then treated with 4 ml of collagenase II (1.4 mg/ml, 270 units/mg, Gibco BRL, New York) at 37° C. for 5 minutes in a moist chamber. Afterwards, the supernatant was removed and the remaining pellet was washed with 10% FBS DMEM. The cells were resuspended into a fresh medium of the same quantity containing 10% FBS. The remaining tissues were treated with fresh collagenase II solution for additional 5 minutes. The incubation process was repeated until the tissue became completely dissolved. The resulting supernatant was centrifuged at 2000 rpm for 2 minutes at the room temperature. The cell pellet was resuspended in 5 ml of cell culture medium, which was then plated on a culture dish and incubated for at least 2 hours at 37° C. inside a 5% CO₂ incubator. The resulting adherent cells are fibroblasts, while the non-adherent cells are myocardial cells. The non-adherent myocardial cells were plated again (5×10⁵ cell/ml) on a 100 mm culture dish and were incubated in α-MEM supplemented with 10% FBS. Afterwards, the cells were cultured in a CO₂ incubator at 37° C. In order to prevent contamination by fibroblasts, α-MEM containing 0.1 mM of 5-bromo-2′-deoxyuridine (Brd-U) (Sigma, MO) was used.

### Inducing Myocardial Infarction and Performing Cell Transplant

All experimental procedures of animal studies have been approved by the Steering Committee for Laboratory Animals of Yonsei University College of Medicine and carried out in accordance with the committee's guidelines for animal protection. Myocardial infarction (MI) was induced on 8-week-old Sprague-Dawley male rats by the vascular occlusion from operating on the left anterior descending (LAD) coronary artery in accordance with the prior art method (Song, S. W., et al. Stem Cells 27, 1358-1365 (2009)). In summary, once Zoletil (20 mg/kg) and xylazine (5 mg/kg) successfully induced anesthesia, the third rib and the fourth rib were cut to open the chest, and the heart was taken out of the body through the intercostal space. The heart was exposed via 2-cm left lateral thoracotomy. The pericardium was cut and a 6-0 suture (Johnson & Johnson) was placed proximal to the left coronary artery below the left atrial appendage. The ligature's end was passed through a short plastic tube to form a noose. To block the coronary artery, the noose was placed against the heart surface directly above the coronary artery, and a hemostat was applied on the noose. After 50 minutes of occlusion, reperfusion was permitted by removing the hemostat and untying the noose to let loose the ligature on the heart surface. For transplantation, the cells were suspended in 30 μl of PBS (1×10⁶ cells), and were injected from the lesion area to the border zones via 30-guage needle Hamilton syringe (Hamilton Co.). During the operation, the animal was ventilated at 95% O₂ and 5% CO₂ with a Harvard ventilator. To label the viable cells as MSCs with DAPI, sterile DAPI solution was added to the culture medium on the transplant-day at a final concentration of 50 μg/ml. The dye was left on the culture plate for 30 minutes. The cells were washed 6 times with PBS, and all excess and unbound DAPI was removed. Labeled cells were desorbed with 0.25% (wt/vol) trypsin and suspended in PBS for transplantation. The operated rats were subjected to morphological analysis in 1 week following the myocardial infarction and to echocardiography test on the third week following the myocardial infarction. The sham-operated group of myocardial infarction-induced rats were injected an identical quantity of PBS only.

### Measuring the Size of the Infarction

TTC staining was used to evaluate the viability of the cardiac muscular tissue and to measure the size of the myocardial infarction. The tissue sections were incubated at 37° C. for 20 minutes in 1% 2,3,5-triphenyltetrazolium chloride (TTC) solution of pH 7.4. The tissue was fixed in 10% PBS-buffered formalin at 4° C. overnight. The heart was cut transversely, and the size of the myocardial infarction was measured as the percentage of the cross-sectional area of infracted left ventricle over the total cross-sectional area of the left ventricle. Both sides of each of the TTC-stained tissue segments were photographed with a digital camera. The infracted zone was measured using Image J 1.40 g software.

### Immunological Analysis of the Host Heart with Transplant Cells

Following the transplant of the cells corresponding to each group, the transplants were sacrificed in multiple intervals after a specific time period and their hearts were cut out. The heart was subjected to perfusion fixation in 10% (vol/vol) neutral buffered formaldehyde for 24 hours, cut transversely into 4 slices of equal thickness, and embedded in paraffin with conventional methods. The 5-μm thick slices were mounted on gelatin-coated glass slides, so that the consecutive slices of tissue cuts through the transplant area may be stained distinctively. Immunological analysis was performed using the manufacturer's instructions manual (Vector Laboratories). In summary, the tissue slices were deparaffinized, rehydrated, and then washed with PBS. Antigen retrieval was conducted via microwaving in 10 mM sodium citrate (pH 6.0) for 10 minutes. The slices were incubated in 3% H₂O₂ to quench any peroxidase that may have been present. The sample was blocked in 2.5% normal horse serum and incubated with primary antibodies (CD31, collagen I, fibronectin, alpha smooth muscle actin). Biotinylated pan-specific universal second antibodies and streptavidin/peroxidase complex reagent were used on the heart slices, and they were stained with antibodies using DAB substrate kit. Counterstaining was performed with 1% Methyl Green, and dehydration was processed with 100% N-butanol, ethanol and xylene. Other slices were analyzed with rabbit anti-connexin 43. For secondary antibodies, FITC-conjugated goat anti-rabbit IgG was used. All images were created with a reflected light fluorescence microscope using excitation filters, and transmitted to a computer equipped with MetaMorph software ver. 4.6 (Universal Imaging Corp). Interstitial fibrosis within the cell transplant was detected via analysis using Masson's trichrome staining and measured with image J 1.40 g software. Morphological changes and inflammatory cell infiltration were measured by performing Hematoxylin and eosin (HE) staining, and the analysis was made by three persons who were unaware of the treatment process.

### Optical Mapping

Adult male laboratory rats (250-300 g) were injected with heparin added with Zoletil (20 mg/kg) and xylazine (5 mg/kg). The heart was cut out, and Tyrode's solution (125 NaCl, 24 NaHCO₃, 1.0 MgCl₂, 4.0 KCl, 1.2 NaH₂PO₄, 5 Dextrose, 25 Mannitol, 1.25 CaCl₂ (in mM)) at pH 7.4 was reverse-perfused in the condition of 95% O₂ and 5% CO₂ through the aorta. Temperature was maintained at 37.0±0.2° C. and the temperature was adjusted to ˜60 mmHg with a peristaltic pump. The heart was marked using voltage-sensitive staining with di-4 NEPPS (Invitrogen), and the stock solution (1 mg/ml of dimethyl sulfoxide, DMSO) was moved through the bubble traps on the aortic cannula. The heart was placed inside a chamber to maintain temperature, and to reduce artificial results due to movement, and 5 mM blebbistatin was added to the aforementioned perfusion solution. The heart was illuminated with quasi-monochromatic light (500±30 nm) using two green LED lamps (LL-50R30-G25, Optronix, Seoul, Korea). For Vm recording, fluorescence was filtered through long-pass filters of cutoff wavelength of 600 nm. Fluorescence images obtained from the anterior surface of the heart were taken with a CCD camera (Model CA D1-0128T, Dalsa, Waterloo, Ontario, Canada) with a spatial resolution of 78×78 mm² per pixel and a maximum time resolution of 490 frames/sec. Field was adjusted to 1.0×1.0 cm² to achieve 128×128 sites simultaneously. Optical recording of the rhythm was continuously monitored by ECGs obtained via widely positioned bipoles (one at the top of the left ventricle, the other high on the sidewalls of the right ventricle) using Biopac System (BIOPAC Systems Inc.). Vulnerability to VT was tested with burst simulations of ventricles performed with stimulation cycle length (S1S1-CL) started at 300 ms and reduced in increments of 10 ms, down to 90 ms. Data were analyzed with commercial software which utilizes Matlab (Mathworks, Natick). Activity and repolarization points of each region were measured from (dF/dt)ₘₐₓ and (d²F/dt²)ₘₐₓ, which appeared to happen at the same time as ˜97% repolarization from the baseline and recovery from the refractoriness. Data were calculated within the spatial domain, and the first-order and second-order derivatives (dF/dt, d²F/dt²) using polynomial filters (3^{rd} order, 13 points) in the time domain. Isochronal maps of activities were generated via previously-taught methods (Choi, B. R. & Salama, G., J Physiol 529 Pt 1, 171-188 (2000)). Conduction velocity at infracted or normal zones was measured with respect to 20 beats, under point stimulation of cycle length of 280 ms. Local conduction velocity vector was estimated by seven nearest pixels of each pixel within the activity time of its temporal wavelength. Distribution of local CV may have high-frequency components such as the collision between motion artifact and wavelengths caused by proximal transmural activities. To improve the SNR (signal-to-noise ratio) of local CV, the local CV was filtered spatially using 7×7 nearest neighbor Gaussian convolution. The dramatic change in size was controlled with log-transformation. The rising time was measured from 10% to 90% of activity-potential amplitude, and low-pass filtered with 1 ms averaging kernel.

### Immunofluorescence

Immmunocytochemical specialization of MSCs was verified as follows. Cells were cultured in 4-well slide chamber, washed with PBS, and incubated in 1% paraformaldehyde solution for 10 minutes. Then, the cells were washed twice with PBS and osmosed into 0.1% Triton X-100 for 7 minutes. Afterwards, the cells were blocked with blocking solution (PBS containing 2% bovine serum albumin and 10% horse serum) for 1 hour, and bound with FITC-conjugated mouse, rabbit and goat antibodies (Jackson Immunoresearch Laboratories) used as secondary antibodies. Then, they were detected with a confocal microscope (Carl Zeiss).

### Sandwich ELISA

100 ng of capture antibodies were bound to polyvinyl chloride (PVC) and microtiter high-binding plate (96 wells) at 4° C. overnight. The plate was washed twice with PBS, capture antibodies were blocked with PBS containing 5% BSA overnight under moist air at the room temperature. The plate was washed twice with PBS, and 5 ug of cell lysates together with blocking buffer was added to each well. The plate was incubated at 37° C. for 1.5 hours. The plate was washed 4 times with PBS containing 0.02% Tween-20. Upon adding detector antibodies, the plate was incubated under moist air and the room temperature for 2 hours, and washed 4 times with PBS containing 0.02% Tween-20. Afterwards, the plate was incubated, with secondary antibodies conjugated with peroxidase diluted 1:1000 with 3% BSA, again at 37° C. for 1.5 hours, and also again was washed 4 times with PBS containing 0.02% Tween-20. Finally, 100 ul of tetramethyl benzidine (TMB) solution (Sigma) was poured as the substrate. After 10 minutes, the reaction was stopped by adding 25 ul of 0.1M H₂SO₄, then the absorbance at 450 nm was immediately measured with an ELISA plate reader (Bio-Rad).

### Western Blot Analysis

The cells were washed once with PBS, and dissolved for 20 minutes in a solution buffer (Cell Signaling Technology) comprising 20 mM tris (pH 7.5), 150 mM NaCl, 1 mM Na2-EDTA, 1 mM EGTA, 1% Triton, 2.5 mM sodium pyrophosphate, 1 mM beta-glycerophosphate, 1 mM Na3VO4, 1 mgml leupeptin, and 1 mM phenylmethylsulfonyl fluoride. Upon centrifuging the cell lysates obtained above at 12000 g for 10 minutes, the supernatant was obtained. The protein concentration was measured using Bradford Protein Assay Kit (Bio-Rad). Quantitative protein was separated with 12% sodium dodecyl sulfate-polyacrylamide gel, and moved with polyvinylidene difluoride membrane (Millipore). The membrane was blocked with tris-buffered saline-Tween 20 (TBS-T, 0.1% Tween 20) containing 5% non-fat dry milk, washed twice with TBS-T, and incubated with primary antibodies (ERK and p-ERK; Santa Cruz Biotechnology) at 4° C. overnight. The membrane was washed 3 times with TBS-T for 10 minutes, and incubated with horseradish peroxidase-conjugated secondary antibodies for 1 hour at the room temperature. After extensive washing, bands were detected with enhanced chemiluminescent reagent (GE Healthcare Life Sciences). The band intensity was quantified using Image J 1.40 g software (NIH).

### RT-PCR

Various gene expressions level were analyzed by Reverse Transcription Polymerase Chain Reaction (RT-PCR). Entire RNAs were extracted with 500 μl of Tri-reagent (Sigma) per 60 mm plate. The sample was voltexed after pouring 100 μl of chloroform on the Tri-reagent. Then, this sample was centrifuged at 12,000 g for 15 minutes at 4° C. There were three layers appeared, the top transparent layer was gathered in a new tube. 250 μl of 2-propanol is added in the above sample afterword, and the mixture in the tube was voltexed for 30 seconds and centrifuged at 12,000 g for 10 minutes at 4° C. Then, the supernatant was thrown away, a pellet was washed with 75% of ethanol (Duksan) which was diluted with diethylpyrocarbonate (DEPC;Sigma). At this point, it was centrifuged at 7,500 g for 5 minutes at 4° C., the pellet was dried for 7 minutes at the room temperature. Finally, 30 μl of water which does not have nuclease was added in the pellet. The quality and quantity of RNA were measured by the OD₂₆₀/OD₂₈₀ with DU 640 spectrophotometer (Effendorf). According to the manufacturer's instruction, complementary DNA was produced by Reverse Transcription System (Promega). 1 μg of total RNA was reversed for 15 minutes at 4° C. with 20 μl of reaction mix comprising of 5 mmol/L MgCl₂, 10 mmol/L Tris-HCl (pH 9.0 at 25° ), 50 mmol/L KCl, 0.1% Triton X-100, 1 mmol/L dNTP, 20 U RNase inhibitor, 0.5 μg oligo-(dT)₁₅ primer, and 10 U reverse transcriptase, then this reaction was terminated by heat for 5 minutes at 99° C. The total volume of PCR mix was 250 μl comprising of 10 pmol/μl of each primer with 200 mM Tri-HCl (pH 8.8), 100 mM KCl, 1.5 mmol/L MgSO₄, 1% Triton X-100, 0.1 mM dNTP and 1.25 U of Taq polymerase. The condition of PCR was as follows: 1 cycle of denaturing for 3 minutes at 94° C., 35 cycles of denaturing for 30 seconds at 94° C., annealing for 30 seconds at 49° C., and extension for 2 minutes 72° C., final extension for 10 minutes at 72° C. RT-PCR product was isolated by electrophoresis in agarose gel, then dyed with ethidium bromide to be visualized.

### Cytokine Detection

The expression level of Cytokine in cell-injected area was measured by rat cytokine array 3.1 (RayBiotech) following the manufacturer's instruction. To extract proteins from tissue, 1×Cell Lysis Buffer was employed. After extraction, the sample was centrifuged, then the supernatant was preserved. Concentration of protein was measured, and the sample was diluted with 2×Cell Lysis Buffer including H₂O. The sample was treated with 2 ml 1×Blocking Buffer to block the membranes by incubating for 30 minutes at the room temperature. The membranes with 1 ml of the sample were incubated for 1-2 hours at the room temperature, and were moved from each container. The membranes were washed 3 times shaking with 2 ml of 1×Wash Buffer II for 5 minutes at the room temperature. 100 μl of biotin-conjugation anti-cytokine was mixed with 1×Blocking Buffer gently and was incubated for 1-2 hours at the room temperature. Wash Buffer I and II were poured 5 times in the Blot, and 1000 times diluted HRP-conjugation streptavidin was injected into the each membrane for 2 hours at the room temperature. Wash Buffer I and II were poured 5 times in the Blot, then the sample was treated with 250 μl of 1×Detection Buffer C and 250 μl of 1×Detection Buffer D for 2 minutes at the room temperature. Finally, the array was exposed to X-ray film, and the signal was detected by chemiluminescence imaging system.

### Surface ECG

Surface 6-lead ECG (lead II is shown by a diagram in the Drawings) was gained for 5 minutes from the rats in a control group and the rats which had been transplanted sham, MSCs, and CPMs. R-R interval, PR interval, QRS duration time, QT and amended QT duration time were measured by continuous evaluations as previously mentioned. All the data were acquired by using Bard stamp amplifier System (C.R. Bard Inc.).

### Systemic Administration of Isoproterenol

2 mg/kg of isoproterenol was injected into the abdominal after attaching radio ECG recorder on a rat. After injection, the frequency of early ventricular systole for 15 minutes was calculated.

### Left Ventricular Catheterization

For invasive hemodynamic study, left ventricular catheterization was conducted between 7 to 11 days after the surgery. Millar Mikro-tip 2 F pressure transducer (model SPR-838, Millar Instruments, Houston, Tex.) was inserted into the left ventricle through the right carotid artery under anesthesia with zoletile (20 mg/kg) and xylazine (5 mg/kg). Real time pressure-volume loops were recorded by a blind examiner, all data were analyzed offline by PVAN 3.5 software (Millar).

### Statistical Analysis

The data about continuous variables were written with average±SE, about variables per categories were written with percentages. The statistical analysis about two groups was conducted by Student's t-test. The examinations about more than two groups were conducted by one-way ANOVA using bonferroni test. The tests about variables per categories were conducted by hi-square test or Fisher exact test. The tests about survival rate were conducted by the Kaplan-Meier method including log-rank test. The significance was considered as P value<0.05.

### Comparative Experimental Example 1

### Confirmation of MSCs Transplantation in Infarcted Myocardium

The effects of transplanting intact MSCs which has not gone through the differentiation-inducing process to infarcted myocardial cell were examined from various angles.

### (1) Study of the Incidence Rate of Sudden Deaths

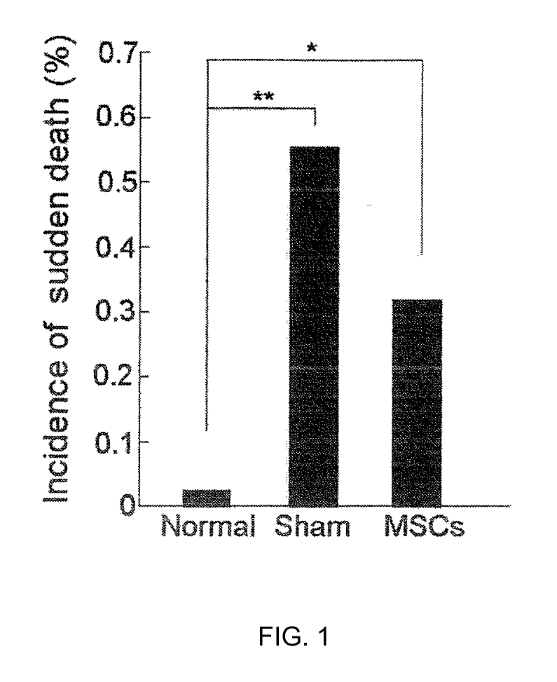

The incidence rate of sudden deaths of MSCs-transplanted group was studied first. FIG. 1 is a graph showing the rate of sudden deaths of control (n=12), sham-operated rats (n=27) and MSCs-transplanted rats (n=19) for 11 days after damage and treatment. As you can see on FIG. 1, MSCs transplantation can reduce the incidence of sudden deaths, however we have noticed it is obviously the best alternative plan to improve the survival rate.

### (2) Immunological Analysis

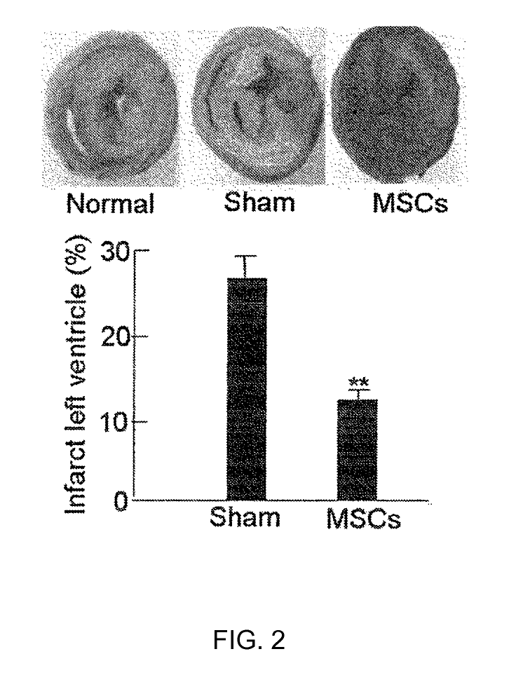

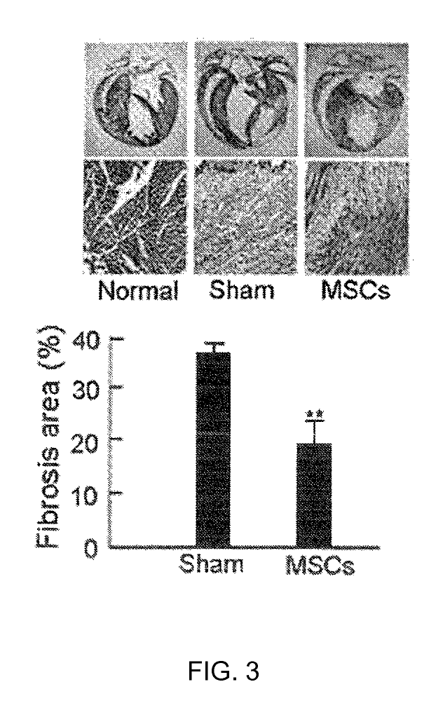

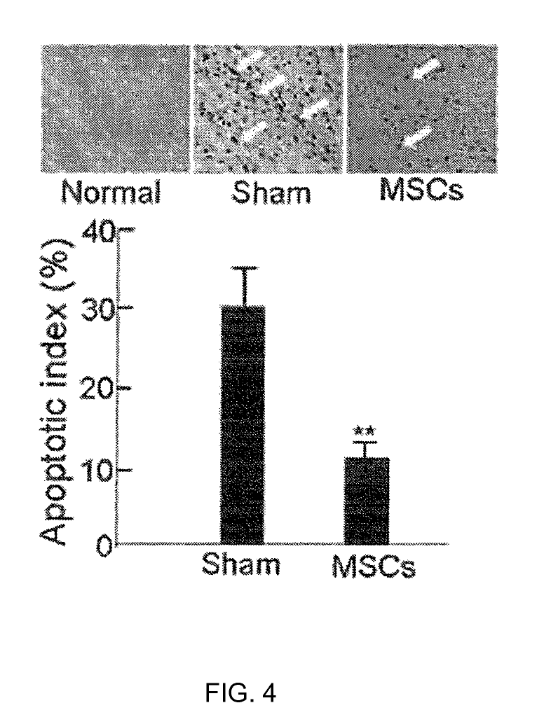

Next, MSCs-transplanted myocardium was examined with the above-mentioned immunological analysis. FIG. 2 demonstrates the result of TTC stain for evaluating viability of myocardial tissue and for measuring the size of the myocardial infarction. As you can confirm on FIG. 2, the infarction size is reduced in MSCs-transplanted domain comparing to sham-injected domain. FIG. 3 demonstrates the result of Masson's trichrome stain for investigating interstitial fibrosis within transplanted cells. It is noticeable that the level of fibrosis, which is mixed with survived myocardium, accelerating re-entry is reduced in MSCs-transplanted domain comparing to Sham-injected domain. Also, FIG. 4 demonstrates the result of TUNEL assay for counting the numbers of cell death caused by apoptosis. The numbers of apoptosis cells induced by ischemia are reduced in MSCs-transplanted domain comparing to Sham-injected domain.

These results are supported by the reports that densely populated fibrosis becomes the enormous physical barrier to cell reproduction (Segers, V. F. & Lee, R. T. Nature 451, 937-942 (2008)), and the direct electrical barrier interfering wavelength delivers which accelerates re-entry (de Bakker, J. M., et al. Circulation 88, 915-926 (1993); Anderson, K. P., et al. J Clin Invest 92, 122-140 (1993)).

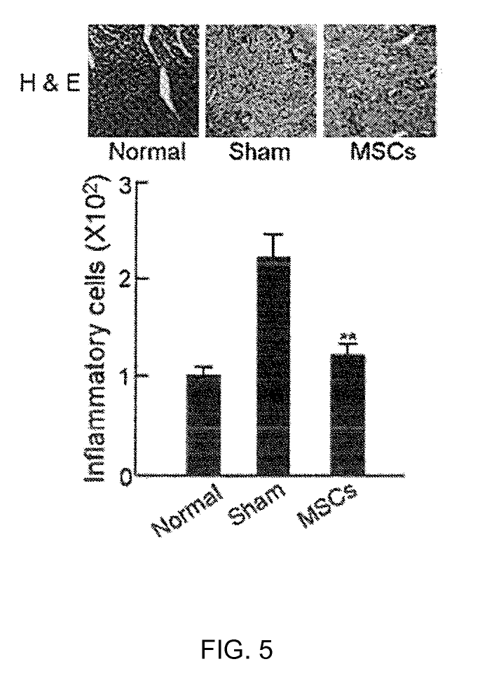

Also, it is known that excessive inflammation can occur not only non-uniform conduction and delayed repolarization (Hoffman, B. F., et al., J Cardiovase Electrophysiol 8, 679-687 (1997); Ishii, Y., et al. Circulation 111,2881-2888 (2005).), but also interference of supplement and survival of precursor cells (Poss, K. D., Wilson, L. G. & Keating, M. T. Heart regeneration in zebrafish. Science 298, 2188-2190 (2002)). Thus, the change of inflammatory cell infiltrate in mesenchymal stem cells transplantation was additionally examined. FIG. 5 demonstrates the result of H& E stain for measuring inflammatory cell infiltrate. The inflammatory cell infiltrate is less on the marginal area of MSCs-transplanted group than the sham-injected group.

### (3) Echocardiograph Analysis

Whether the LV function is improved or not in mesenchymal stem cells transplantation was examined by transthoracic echocardiography. As a result, as you can see in Table 1 below, it has been confirmed that the contraction phase performance and the LV function measured by transthoracic echocardiography were improved in the MSCs-transplanted rats.

TABLE 1

| Variables | Normal (n = 8) | Sham (n = 8) | MSCs (n = 8) |
|---|---|---|---|
| Heart rate (bpm) | 236.8 ± 12.30 | 260 ± 13.47 | 253 ± 8.98 |
| LVEDD (mm) | 5.67 ± 0.23 | 7.64 ± 1.27** | 6.82 ± 0.77 |
| LVESD (mm) | 3.80 ± 0.22 | 6.90 ± 1.15** | 5.92 ± 0.61^{†} |
| FS (%) | 35.99 ± 2.63 | 9.79 ± 1.05** | 13.08 ± 2.11^{†} |
| LVESV (ml) | 0.43 ± 0.05 | 1.03 ± 0.42** | 0.74 ± 0.25^{†} |
| LVEDV (ml) | 0.13 ± 0.01 | 0.78 ± 0.32* | 0.49 ± 0.15^{†} |
| LVEF (%) | 70.59 ± 4.41 | 24.74 ± 2.39** | 32.29 ± 4.62 |

In the above Table, the figures is shown with average±SE
Each *p<0.01, **p<0.001 vs. Normal; Each ⁺p<0.01, ⁺⁺p<0.001 vs. Sham
Abbreviation: LVEDD (left ventricular end diastolic diameter); LVESD (left ventricular end systolic diameter); FS (fractional shortening); LVEDV (left ventricular end diastolic volume); LVESV (left ventricular end systolic volume); LVEF (left ventricular ejection fraction).

However, in spite of this immunological and functional improvement, we noticed most of transplanted MSCs still did not express cardiac troponin T (cTnT) after 7 to 11 days since the transplantation. This indicates that they were not differentiated to cardiogenic cells, so they could induce returning arrhythmia by working as current-sink.

### (4) Electrical Stability Analysis

Thus, we have additionally examined in sham-operated heart and stem cell-transplanted heart as a control group after 7 to 11 days since damage and treatment about how MSCs affect to electrical stability in infracted heart by optical mapping using Langendorff perfusion and electrical vinerability test.

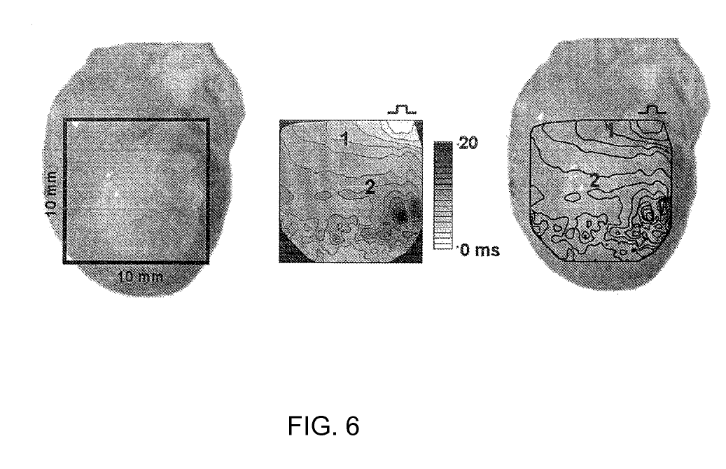

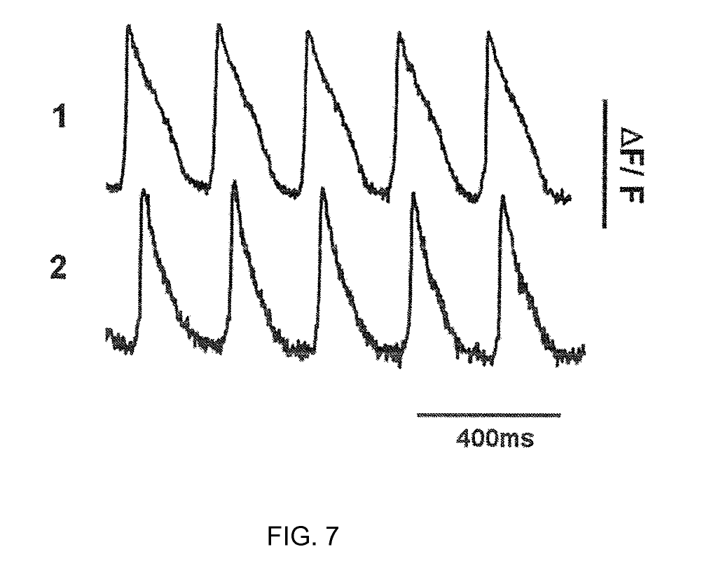

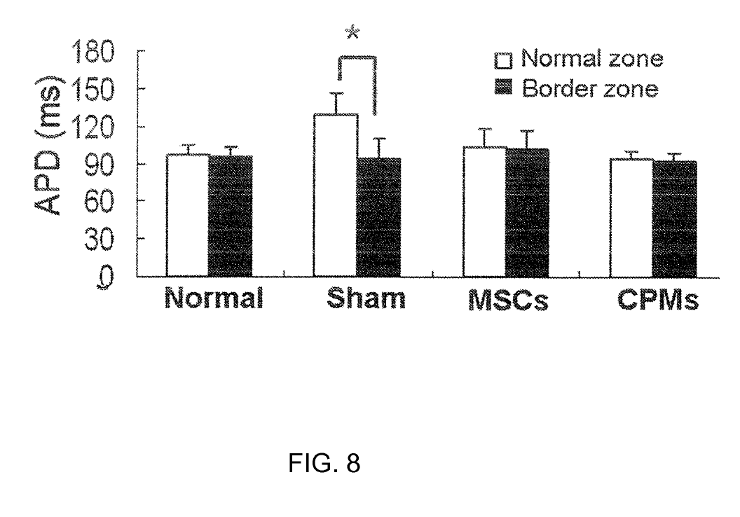

FIG. 6 shows real images (left panel) of infracted heart. It clearly shows the blue-colored change in the infarcted area, the border zone (2) occurring slow radio waves is confirmed by the active map (middle and right panel). FIG. 7 demonstrates action potential of control, sham-operated, MSCs- and CPMs-transplanted hearts. Ectopic beats are more suppressed in the MSCs-transplanted hearts under sinoatrial node rhythm and electrical stimulations, unlike the present of frequent spontaneous focal activity from the border zone of the infracted myocardium that typify the arrhythmia mechanism of myocardial infarction (sham-operated group 46%, n=13, MSCs-transplanted group 22%, n=9). Furthermore, as we can see in FIG. 8, while the action potential durations (APDs) reported from MSCs-transplanted area are similar to the un-infarcted area (101.9±14.9 ms vs. 103.5±14.1 ms in MSCs-transplanted heart, 92±7.4 mm/ms vs. 93.2±7.1 mm/ms, p>0.05 in Sham-transplanted heart), the border zone of sham-operated heart has shorter APDs than normal area (93.9±16.2 ms vs. 128.8±18 ms, *p<0.05).

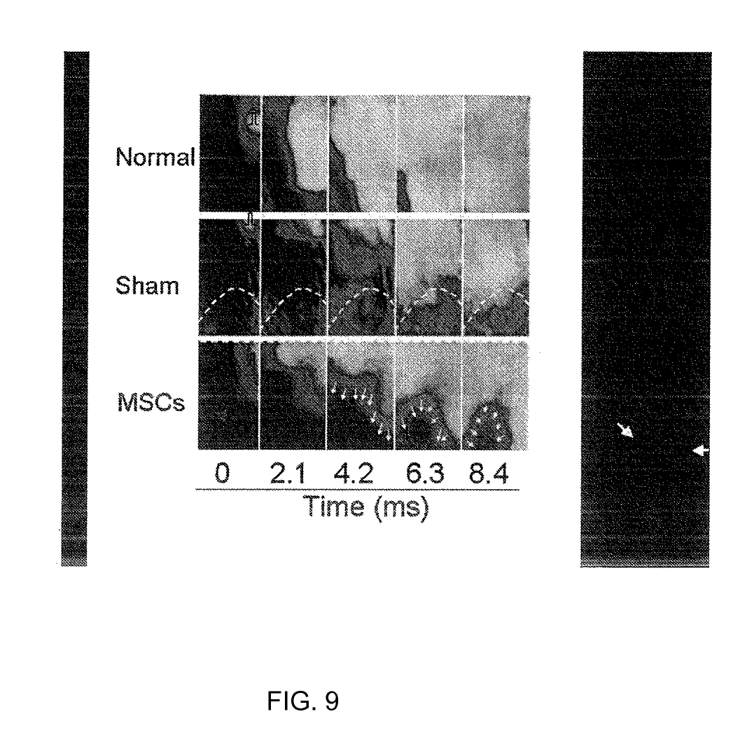

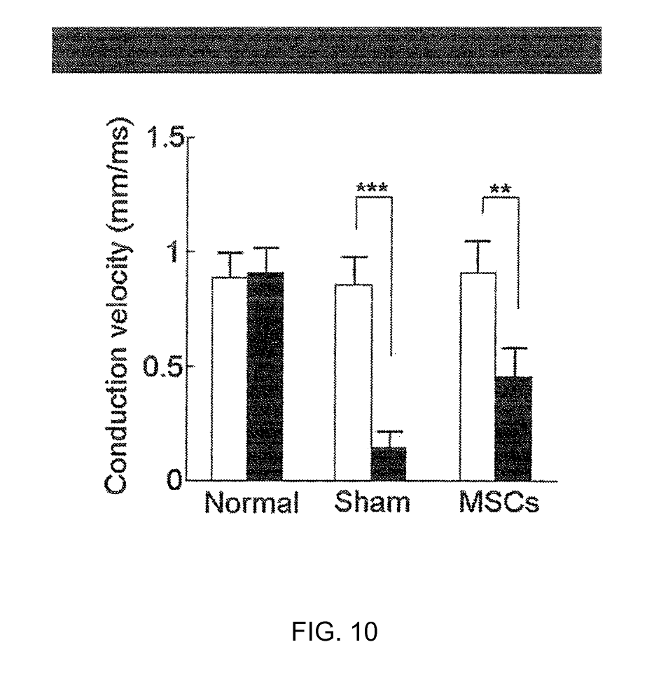

However, as we can see in FIG. 9, APDs is spread into the infarction zone, but it was slow and non-uniform in the MSCs-transplanted heart. It indicates that the conditions which are needed to draw stable circuit movement are not removed (Takahashi, T., et al. Heart Rhythm 1, 451-459 (2004)). FIG. 10 is a graph showing a localized conduction velocity (CV) of sham-administered heart and MSCs-transplanted heart. The CV measured in the border zone in Sham-operated hearts is reduced comparing to the un-infarcted area (n=8, 0.14±0.1 mm/ms vs. 0.91±0.07 mm/ms, ***p<0.0001). MSCs-transplanted lesion is 3 times higher than sham-operated lesion, still the CV of MSCs-transplanted lesion is lower than the normal area(n=7, 0.45±0.1 mm/ms vs. 0.91±0.1 mm/ms, **p<0.001). As we can confirm from FIG. 10, the local Conduction Velocity (CV) from the activation point of active potential is still low in MSCs-transplanted area comparing to other un-infarcted area. It indicates that this results from the nature of mesenchymal stem cells which does not react to the stimulation, and their abilities of working as current-sink (Chang, M. G., et al. Circulation 113, 1832-1841 (2006); Beeres, S. L., et al. J Am Coll cardiol 46, 1943-1952 (2005)).

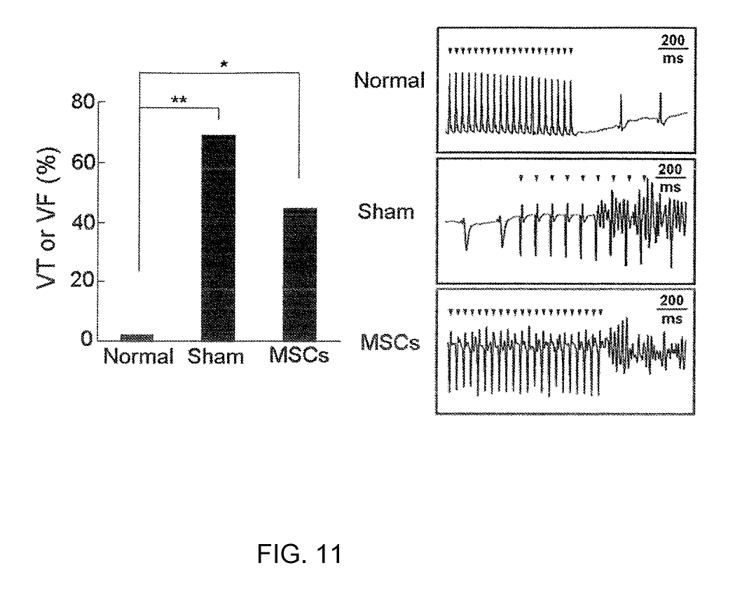

FIG. 11 shows the result of electrical vulnerability test using the burst pacing protocol. The graph on the left panel of FIG. 11 shows the inductivity of Ventricular Tachycardia (VT) or Ventricular Fibrillation (VF) in Normal, sham-operated group and MSCs-transplanted group. The susceptibility is significantly increased about VT or VF inductivity in sham-operated group (n=13) comparing to control group (n=12) (69.2% in sham vs. 0% in control group, p=0.0005). The VT inductivity seems to be decreased in MSCs transplantation (n=9) comparing to sham-operated group (44.4% in MSCs-transplanted group vs. 69.2% in sham-operated group, p=0.38), fatal ventricular tachyarrhythmia still happens frequently even after MSCs transplantation (44.4% in MSCs-transplanted group vs. 0% in control group, p=0.017). The right panel in FIG. 11 shows a typical example of ECG in electrical vulnerability ex vivo test of burst pacing protocol. Burst stimulations of 220 ms Cycle Length (CL) induced VT in the sham-operated hearts. During reducing CL to 90 ms by stages, VT in the MSCs-transplanted heart was easily induced at 100 ms of CL, however VT in the control group was not induced at all even at 90 ms of CL. The arrows indicate electrical stimulations.

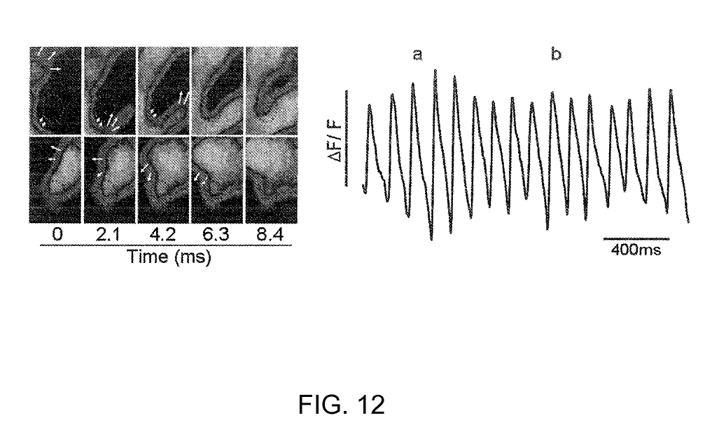

FIG. 12 shows sequential voltage map of ventricular tachycardia (VT) of MSC-transplanted area. The Single re-entry developing spiral wave length is slowly spread into MSCs-injected area (red circle) (upper figure of the left panel), then fixed in the MSCs-injected area (bottom figure of the left panel).

The white arrows in here indicate the directions of the wave front. The right panel shows the active potential in visual recording. This shows the result that the electrical flow is poor around the MSCs-injection site.

### Experimental Example 1

### Confirmation of Property of the Cardiogenic Cells Differentiated from PTK-Inhibited MSCs (CPMs)

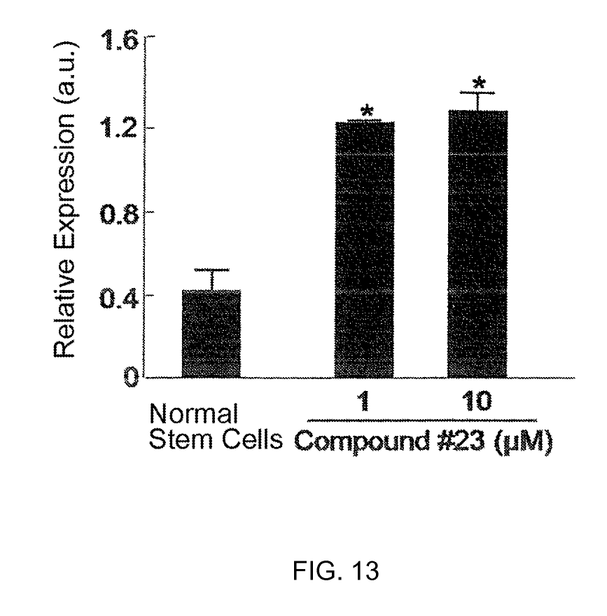

The present inventors continued to investigate the ways to induce mesenchymal stem cells into cardiogenic cells, and study to screen the compounds for inducing mesenchymal stem cells to differentiate into cardiogenic cells. These studies brought the result that the compound #23(N-(3-bromo phenyl)-6,7-diethoxyquinazoline-4-amine) which is PTK inhibitor, namely the compound of Formula 1 can differentiate and induce mesenchymal stem cells to cardiogenic cells. FIG. 13 shows the result of sandwich ELISA where differentiation into myocardial cells increases depending on the concentration of compound #23 treated. As we can see in the FIG. 13, it is confirmed that PTK inhibitor induces mesenchymal stem cells to cardiogenic cells as increasing expression ratio of cardiac troponin T (cTnT) by the method dependent on dosage.

MSCs has various potential advantages for heart recovery comparing to other stem cells, however it still faces few problems to solve at the preclinical phase. Although precondition of MSCs including genetic transformations has been conducted to increase its treatment effect, most of the distinct concern is about how the transplanted MSCs can complete the electro-mechanical integration with the host tissues. Our result shows first that the new cell type originated from MSCs overcomes the best alternative prevention of sudden-death caused by intact MSCs after transplantation, and provides new strategy to improve electro-mechanical integration in the cellular-based treatments about myocardial infarction. And to conclude, the transformation of MSCs to myocardial cells by PTK inhibitor, which helps MSCs to be in balance with host tissues electro-mechanically after transplantation, can be the best treatment strategy for clinical applications of the MSCs about infarcted myocardium.

1. A method for inducing mesenchymal stem cells to differentiate into cardiogenic cells, comprising treating mesenchymal cells with a Protein Tyrosine Kinase (PTK) inhibitor

2. The method of claim 1, wherein the PTK inhibitor is represented as a compound of formula (I): 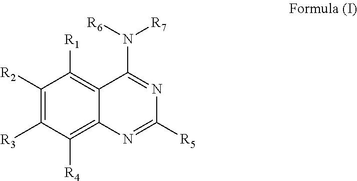
wherein:
R₁, R₂, R₃, R₄, R₅ and R₆ are each independently H, Cl_{—12} alkyl, C₁₋₁₂ alkoxy, hydroxyl, carboxyl or halogen atom; and
R₇ is C₆₋₁₂ aryl, wherein said C₆₋₁₂ aryl is unsubstituted or substituted with more than one substituents selected from the group consisting of C₁₋₁₂ alkyl, C₁₋₁₂ alkoxy, hydroxyl, carboxyl and halogen atom.

3. The method of claim 2, wherein
R₁, R₂, R₃, R₄, R₅ and R₆ are each independently H, C₁₋₄ alkyl, or C₁₋₄ alkoxy; and R₇ is phenyl, wherein said phenyl is unsubstituted or substituted with more than one substituents selected from the group consisting of C₁₋₄ alkyl, C₁₋₄ alkoxy, hydroxyl, carboxyl and halogen atom.

4. The method of claim 2, wherein
R₁, R₂, R₃, R₄, R₅ and R₆ are each independently H, or C₁₋₄ alkoxy; and
R₇ is phenyl, wherein said phenyl is unsubstituted or substituted with C₁₋₄ alkyl, C₁₋₄ alkoxy, or halogen atom.

5. The method of claim 1, wherein the PTK inhibitor is N-(3-bromophenyl)-6,7-diethoxyquinazoline-4-amine.

6. The method of claim 1, wherein the mesenchymal cells are obtained from bone marrow, tissues, embryo, cord blood, blood or body fluid.

7. The method of claim 1, wherein treating the mesenchymal stem cells with the PTK inhibitor is done by culturing the mesenchymal stem cells in a medium containing the PTK inhibitor.

8. The method of claim 7, wherein the culturing lasts for 5 to 15 days.

9. The method of claim 1, wherein the cardiogenic cells have an increased expression of a cardiac specific marker compared to that of the mesenchymal stem cells.

10. The method of claim 9, wherein the cardiac specific marker is selected from the group consisting of cardiac troponin T, myosin light chain and myosin heavy chain.

11. The method of claim 1, wherein the cardiogenic cells have an increased expression of connexin 43 compared to that of the mesenchymal stem cells.

12. The method of claim 1, wherein the cardiogenic cells have an increased expression of a Ca²⁺ homeostasis-related protein compared to that of the mesenchymal stem cells.

13. The method of claim 12, wherein the Ca²⁺ homeostasis-related protein is SERCA 2a or LTCC.

14. A composition for inducing differentiation of mesenchymal stem cells into cardiogenic cells, wherein the composition contains a protein tyrosine kinase (PTK) inhibitor.

15. The composition of claim 14, wherein the PTK inhibitor is represented by a compound of formula (I): 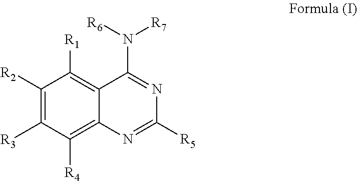
wherein:
R₁, R₂, R₃, R₄, R₅ and R₆ are each independently H, C₁₋₁₂ alkyl, C₁₋₁₂ alkoxy, hydroxyl, carboxyl or halogen atom; and
R₇ is C₆₋₁₂ aryl, wherein said C₆₋₁₂ aryl is unsubstituted or substituted with more than one substituents selected from the group consisting of C₁₋₁₂ alkyl, C₁₋₁₂ alkoxy, hydroxyl, carboxyl and halogen atom.

16. The composition of claim 15, wherein
R₁, R₂, R₃, R₄, R₅ and R₆ are each independently H, C₁₋₄ alkyl, or C₁₋₄ alkoxy; and R₇ is phenyl, wherein said phenyl is unsubstituted or substituted with more than one substituents selected from the group consisting of C₁₋₄ alkyl, C₁₋₄ alkoxy, hydroxyl, carboxyl and halogen atom.

17. The composition of claim 15, wherein
R₁, R₂, R₃, R₄, R₅ and R₆ are each independently H, or C₁₋₄ alkoxy; and
R₇ is phenyl, wherein said phenyl is unsubstituted or substituted with C₁₋₄ alkyl or C₁₋₄ alkoxy.

18. The composition of claim 14, wherein the PTK inhibitor is N-(3-bromophenyl)-5,7-diethoxyquinazoline-4-amine.

19. A pharmaceutical composition for treating heart diseases, comprising inducing differentiation of mesenchymal stem cells into cardiogenic cells according to the method of claim 1.

20. The pharmaceutical composition of claim 19, wherein the heart diseases include myocardial infarction, cardiac insufficiency, or arrhythmia.
